(19) United States
(12) Patent Application Publication
(10) Pub. No.: US 2008/0140032 A1
(43) Pub. Date: Jun. 12, 2008

(54) Urine Receptacle Holder

(76) Inventor: Martin J. O'Malley, Westminster, CO (US)

Correspondence Address: Leyendecker & Lemire, LLC, C/O PORTFOLIO IP, P.O. BOX 52050, MINNEAPOLIS, MN, 55402, US

(21) Appl. No.: 11/567,288

(22) Filed: Dec. 6, 2006

(51) Int. Cl. A61M 1/00; B65D 25/28

(52) U.S. Cl. 604/322; 220/737

(57) ABSTRACT

An embodiment of a urine test receptacle holder comprises a unitary polymeric material having a first section, a third section, and an second section. The third section is typically comprised of a bore, which is typically adapted to allow a urine receptacle such as a cup, to fit within. The third section is integrated to the distal end of the second section, with the second section typically angled up and away from the third section in a 45 degree angle. The proximal end of the second section typically integrated to the first section, also in a 45 degree angle. The first section is adapted to allow a person other than the person providing the urine, to hold onto the holder and coupled receptacle in a position that allows the receptacle to receive urine from a female.

### FIELD OF THE INVENTION

This invention generally relates to holders of urine collection devices.

### BACKGROUND

Urine analysis is a fact of life for many people. Whether a urine test is required while interviewing for a job, or if a urine sample is required as part of a professional, amateur, or student athlete's requirement to continue playing a sport, many people are required to provide a medical health professional with a urine sample. Additionally some persons are required to take an urinalysis test as part of an incarceration program. Overall, urinalysis is highly prevalent in today's society and as can be expected, quite frequently the examinee of a urinalysis exam is female. Particular issues arise when females are required to take a urine test.

For example, when a female is required to provide a urine sample, particularly in the case of an incarcerated female, an attendant is required to watch the female produce the urine sample in order to ensure the sample's validity. In the past, women have been known to supply the urine of another through inserting a vial of urine into their vagina, and subsequently releasing the urine when asked to so by the attendant. Due to moral, practical, and statutory reasons, the urination collection device—typically a cup with an attachable lid, is typically held in place by the person required to provide the urine.

Recently, however, different types of urination collection devices have been developed in order to increase the reliability that the urine is unaltered and is an actual urine specimen from the person required to provide a specimen and is not a vaginally produced or otherwise altered specimen. For example, U.S. Pat. No. 7,011,634 describes a urination collection device wherein the device rests on the toilet seat. These types of devices are impractical at times and do not allow an attendant to ensure the urine is actual urine produced by the person required to provide a specimen.

In order to ensure that the urine provided for a urination analysis examination is actual urine produced by the person undergoing the urination test, under many circumstances, the attendant is required to hold the urination collection device. For reasons such as cleanliness and to ensure that no unwanted items are placed into the urine, devices have been developed which an attendant may hold the urination collection device such as cup in the correct position, yet are also able to keep the attendant substantially free from contacting any urine which misses the urination collection device. One such device a planar plexiglass device, at least a portion of which is about ¾ of an inch thick, 12 inches long, and wide enough to hold a standard urination collection cup in a hole located proximate to one end of the device. Each portion of the device is located in a single plane, thereby making it difficult for a urination exam attendant to position the urine receptacle opening in a substantially horizontal manner.

One other problem with such a prior-art device is the inability of the attendant to ensure the person undergoing the urine test is providing her actual urine and not providing urine from a vaginally-inserted urine receptacle. This is due to the fact that the prior-art plexiglass urination device is a straight device and does not allow an attendant to properly view the collection of the urine while holding the device. An additional problem with the current devices is that they must be cleaned and reused after every urination gathering occurrence since the prior art device is not adapted to be used a single-use device.

### SUMMARY OF THE DRAWINGS

FIG. 1A is an isometric view of one embodiment of the invention holding a urination collection device with an attached lid.

FIG. 1B is a side view of a portion of one embodiment of the invention holding a collection device with an attached lid.

FIG. 2 is a side view of one embodiment of the invention.

FIG. 3 is a top view of one embodiment of the invention.

FIG. 4 is a top view of one embodiment of the invention having a clasp third section.

FIG. 5 is a top view of one embodiment of the invention.

FIG. 6 is a view of one method of collecting urine.

FIG. 7 is a view of a bottom surface with a ridged portion of one embodiment of the invention.

### DETAILED DESCRIPTION

The current invention is a urine receptacle holder adapted to allow the attendant of a urination examination to view the acquisition of the urine while the urine collection device holder is coupled to a receptacle; and subsequently dispose of the holder after the collection of the urine sample is completed. The device may be held either by the person providing the urine sample or the examination attendant, with a self-held holder being used in self-administered urine collection scenarios, among others. Generally, the device allows the examination attendant to use a new holder (whether sterile or not) for every use since the device is adapted to be used as a single-use disposable device.

The inexpensive polymeric material used in one embodiment eliminates the need to clean the urine collection device holder after every use since the holder is simply disposed of after each use. Such disposal may amount to placing all holders into a special bin which is sent to a recycle center with or without other recyclable plastic articles. Typical disposal contemplates placing the holder in a trash bin. In one embodiment, the unique angular shape of the device provides an attendant the ability to view the acquisition of the urine, yet providing the examination subject a degree of privacy, while at the same time the attendant holds the device. Or, the urine provider holds the device herself while the attendant views the urine entering the urine receptacle.

As one feature of the holder which is an improvement over prior art holders is the disposability of the holder, the material that the holder is made out of is typically a low-grade plastic. Any type of plastic may be used, but for economical purposes, recycled commodity plastic is typically used. Recycled plastic labeled Type 1 through Type 6 by the Society of the Plastics Industry (“SPI”) may be used to create the holder. Typically, the resulting material of the holder may be a polyethylene material such as, but not limited to, a Type 2 HDPE High-Density Polyethylene. However, the holder may be made of a material such as, but not limited to, a polypropylene or any other type of suitable material. For instance, variations are also contemplated wherein the holder is comprised of a compressed paper product.

Whatever type of material is used to create the holder, the holder is parallel with the floor when a first section is also parallel with the floor. Typically, the holder is comprised of 3 sections—(i) a first section being a holding section; (ii) a second section being an angled section; and (iii) a third section being a support section. The first and third sections are generally parallelly disposed relative to each other, with the second section being angled therebetween.

The first section is typically adapted to allow a person, such as an urination examination attendant or the person providing the urine, to hold onto the holder while the urination receptacle is filled up with urine, yet allow the attendant to be substantially free from contacting any urine. The first section is typically a generally flat section. In one embodiment, one surface of the first section, such as the top surface, is a textured such as with a criss-cross indentation to enable the person holding the holder to have a better grip than if the surface was flat and smooth.

The first section in one embodiment is a generally rectangularly-shaped section, but the first section may be another shape as well. A preferred length of an embodiment's first section is about 1.25 inches. A preferred width of one embodiment's first section is about 1 inch and a preferred thickness of one embodiment's first section is about 0.140 inches along the ridged edge of the section and about 0.085 inches in an unridged center portion (or web portion). The particular length and width provides a user with ample area to grasp while the total amount of plastic material utilized is minimized. The use of a ridged edges on this section and other sections permits the construction of a holder that uses a relatively small amount of plastic while maintaining a suitable stiffness to adequately support the urine receptacle even when filled. The first section is integrated to a proximal end of the second section typically along a first section edge that runs width-wise.

In one embodiment, the second section angles in a downward direction away from the first section, with the top surface of the second section being angled about 225 degrees relative to the top surface of the first section. This angle is typically the preferred angle in order to provide the greatest support to the urination receptacle before, during and after the urination receptacle is filled with urine. Additionally, the 225 degree angle between the two top surfaces typically allows the attendant to easily view the receptacle being filled up. Having an angled second section allows the second section to be thinner than an unangled second section would have to be thereby reducing the amount of plastic required for the second section and the holder.

A most preferred length of the second section is about 5.25 inches, while a most preferred width of the second section in one embodiment is about 0.75 inches. A portion of the second section typically has a larger width than the rest of the second section—where the second section is integrated to the first section. In one embodiment, the width of the second section tapers from the first section integration of 1 inch to ¾ of an inch. The thickness of the second section is about 0.095 inches in the ridged portion that extends along the section's edge and about 0.65 inches in an unridged web portion that spans between the opposing ridged portions.

A distal end of the second section is typically integrated to the third section. In one embodiment, the top surface of the second section is typically at an angle which is 135 degrees from the top surface of the third section. Therefore, the first section and the third section are typically substantially parallel. The third section is typically comprised of 2 portions—(i) an extension portion; and (ii) an attachment portion. The attachment portion in one embodiment typically comprises a bore, but the attachment portion may also be a clasp or any other mechanism adapted to couple to a urination receptacle such as, but not limited to, a cup. The thickness of the third portion is about 0.110 inches in a ridged portion and about 0.085 inches in an unridged portion.

Generally, the urine receptacle holder is adapted to allow an attendant to a urination examination to hold a urination receptacle in place for a female examination subject in order to verify that the urine has not been tampered with. To do so, in one method, the urination receptacle is coupled to the holder by either placing the cup through a bore up to a cup lip such that the lip is adapted to keep the cup from passing through the bore. Additionally, the cup or other receptacle may couple to the holder through means of a clasp in one variation.

Upon coupling the urination receptacle to the holder, in one variation, an attendant holds onto the first section of the holder and positions the receptacle in a place adapted to collect urine from a female upon release of the fluid. A variation may also allow the examination subject herself to hold onto the first section of the holder and position the receptacle in a position adapted to allow the receptacle to receive urine. Additionally, in one variation, the receptacle is typically located in a position that allows the attendant to view the receptacle as it is being filled up with urine.

Upon the receptacle receiving urine, the attendant in one variation removes the holder and coupled receptacle from the position adapted to receive urine. The attendant may thereupon place a lid on the cup and uncouple the cup from the holder. The holder may then be disposed of.

The holder has a bottom surface. The bottom surface typically has a ridged portion in one variation. The ridged portion is typically located proximal the intersection of the bottom surface with one or more side surfaces. The ridged portion creates a variation less flexible than an embodiment that has the same amount of plastic but does not have a ridged portion. Therefore, the ridged portion creates stiffness in a variation. A stiffer variation helps ensure the embodiment adequately holds the opening to a coupled urine receptacle in a substantially horizontal position when the holding section is in a substantially horizontal position.

### Terminology:

The terms and phrases as indicated in quotation marks (“ ”) in this section are intended to have the meaning ascribed to them in this Terminology section applied to them throughout this document, including in the claims, unless clearly indicated otherwise in context. Further, as applicable, the stated definitions are to apply, regardless of the word or phrase's case, tense or any singular or plural variations of the defined word or phrase.

The term “or” as used in this specification and the appended claims is not meant to be exclusive rather the term is inclusive meaning “either or both”.

References in the specification to “one embodiment”, “an embodiment”, “a preferred embodiment”, “an alternative embodiment”, “a variation”, “one variation”, and similar phrases mean that a particular feature, structure, or characteristic described in connection with the embodiment is included in at least an embodiment of the invention. The appearances of phrases like “in one embodiment”, “in an embodiment”, or “in a variation” in various places in the specification are not necessarily all meant to refer to the same embodiment or variation.

The term “couple” or “coupled” as used in this specification and the appended claims refers to either an indirect or direct connection between the identified elements, components or objects. Often the manner of the coupling will be related specifically to the manner in which the two coupled elements interact.

Directional and/or relationary terms such as, but not limited to, left, right, nadir, apex, top, bottom, vertical, horizontal, back, front and lateral are relative to each other and are dependent on the specific orientation of a applicable element or article, and are used accordingly to aid in the description of the various embodiments and are not necessarily intended to be construed as limiting.

As applicable, the terms “about” or “generally” as used herein unless otherwise indicated means a margin of +−20%. Also, as applicable, the term “substantially” as used herein unless otherwise indicated means a margin of +−10%. It is to be appreciated that not all uses of the above terms are quantifiable such that the referenced ranges can be applied.

The term “integrate” or “integrated” as used in this specification and the appended claims refers to a blending, uniting, or incorporation of the identified elements, components or objects into a unified whole.

### First Embodiment of a Urination Collection Device Holder:

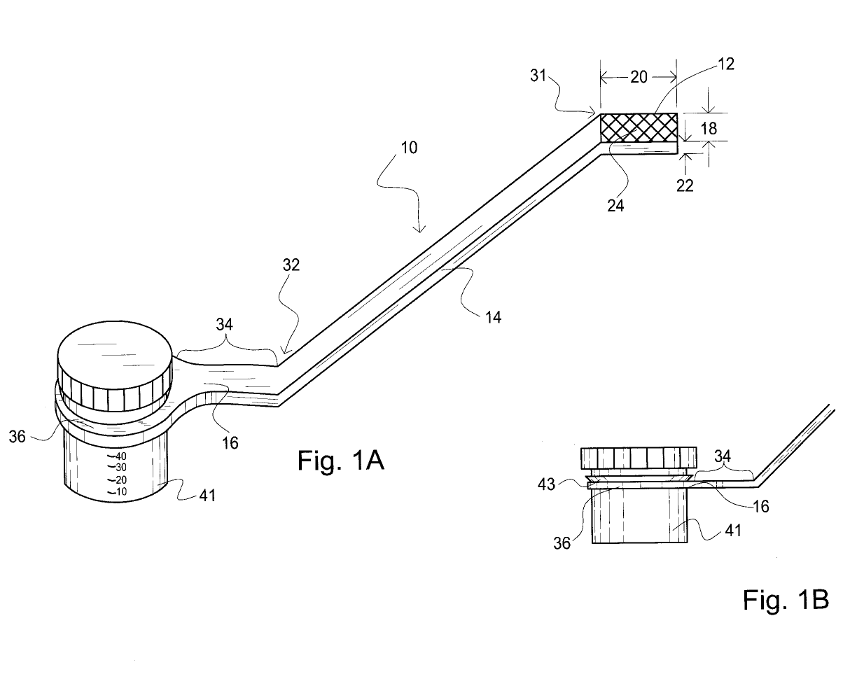

As shown by FIGS. 1 through 5, one embodiment of a urination collection device holder 10 includes a first section 12, an second section 14, and a third section 16. The holder 10 is typically comprised of a single unitary material, although variations may not be unitary. An embodiment's material is typically a polymeric material, with one embodiment being comprised of a recycled plastic and/or commodity plastic.

A recycled plastic embodiment typically allows the holder 10 to be manufactured at a greatly reduced cost, which may be passed along to any potential purchaser. Having a lower purchase cost may allow the holder to be purchased in bulk quantities. Through bulk purchasing, the holder may be able to be used as a single-use device, with any user of the holder able to throw away the holder after a single use. If the device is made of a recyclable material such as, but not limited to, recyclable polyethylene, with the polyethylene being Type 2 polyethylene in one embodiment, some users may prefer to send the holder to a plastics recycling center, potentially along with other recyclable plastic material, instead of throwing the holder away after each use. Typically, the embodiment is made from an injection molding process. However, the embodiment may be cut, heated, and shaped, or may be made from any other suitable process. For instance, embodiments of the holder could be vacuum molded from plastic sheet material.

The first section 12 is adapted to allow a person to grip the holder 10, typically between a thumb and forefinger. Therefore, the first section is adapted to allow a person to grab onto a holder which is coupled to an urination collection device/receptacle 41 containing urine. For example, in one embodiment, a preferred width 18 of the first section may be about 1 inch; the length 20 of the first section is about 1.25 inches to 1.75 inches, with a thickness 22 of the first section being about ⅛of an inch. Typically, at least one surface of the first section in one embodiment is a textured surface. As best shown in FIG. 1, the textured surface may be a top surface 24 and the textured portion may be diagonal indented lines which are “criss-crossed” in the top surface. The first section may generally resemble another non-rectangular shape, such as, but not limited to, an oval shape having dimensions generally similar to a rectangular-shaped first section.

The first section 12 in one embodiment is integrated to the second section 14 at an second section proximal end 31. In one embodiment, the first section is integrated to the second section along a widthwise portion of each section. Typically, the width 28 of the second section decreases over a portion 26 of the second section. The portion of the second section possessing decreasing width typically extends from the first section integration point to a point on the second section distal the first section integration point.

In one embodiment, the length of the portion 26 of the second section 14 where the width decreases is about ½ an inch in length. The width may decrease from the width of the first section, which is typically 1 inch in one embodiment, to the width of the second section, which is typically ¾ of an inch in one embodiment. The length 30 of the second section in one embodiment is longer than the first section length, with the second section length being about 5¼ inches. Therefore, in an embodiment comprised of decreasing width portion 26, the length of the portion of the second section that does not decrease in width and has a substantially uniform width may generally be 4¾ inches long. In one embodiment, the length of the second section is 3.5 times the length of the first section. The second section may be 1.5 times the length of the third section as well. The thickness of the second section is typically the same thickness as the first section 12 in one embodiment. In other embodiments, the thickness between sections varies.

Similar to the first section 12, the second section 14 in one embodiment is typically integrated to the third section 16. However, an embodiment may be comprised of coupled sections instead of integrated sections. For example, the sections may couple together through a mechanism such as, but not limited to, a snapping mechanism. In one embodiment, the third section is comprised of an extension portion 34 and an attachment portion 36. However, an embodiment may only be comprised of an attachment portion.

The extension portion 34 is typically a part of the third section 16 in one embodiment that is integrated to the second section 14. The extension portion has substantially the same width and thickness as the second section. Additionally, a proximal widthwise end of the extension portion is typically integrated to a distal end 32 of the second section. A distal widthwise edge of the extension portion is typically integrated to the attachment portion 36.

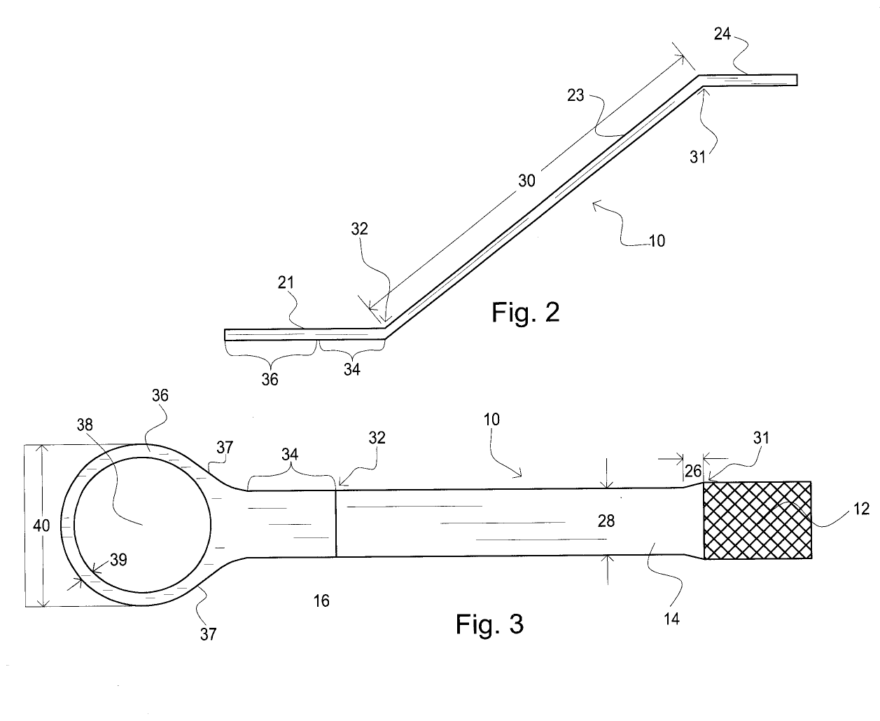

The attachment portion 36 in one embodiment is typically comprised of a bore 38. In one embodiment, the bore is a bore having a generally circular cross-section. As best shown by FIGS. 2 and 3, the attachment portion extends away from the integrated extension portion 34 in substantially the same plane that the extension portion is located in. The width 40 of the attachment portion typically varies, with the attachment portion width typically initially expanding as it extends away from the extension portion. When an outer edge 37 of the extension portion is proximal the bore, the outer edge of the extension portion typically remains substantially an equal distance from the bore. For example, the thickness 39 of the distance between the attachment portion outer edge and the bore is generally equal to the thickness of the ridged portion of the attachment portion. In one embodiment, this thickness is ⅛ inch.

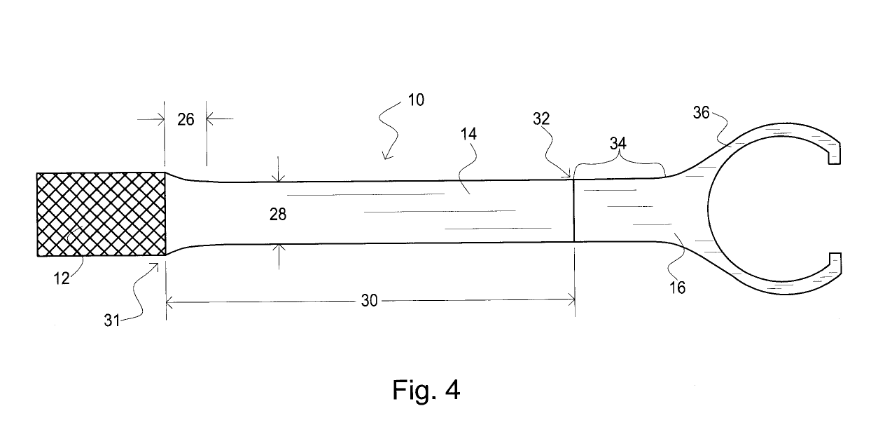

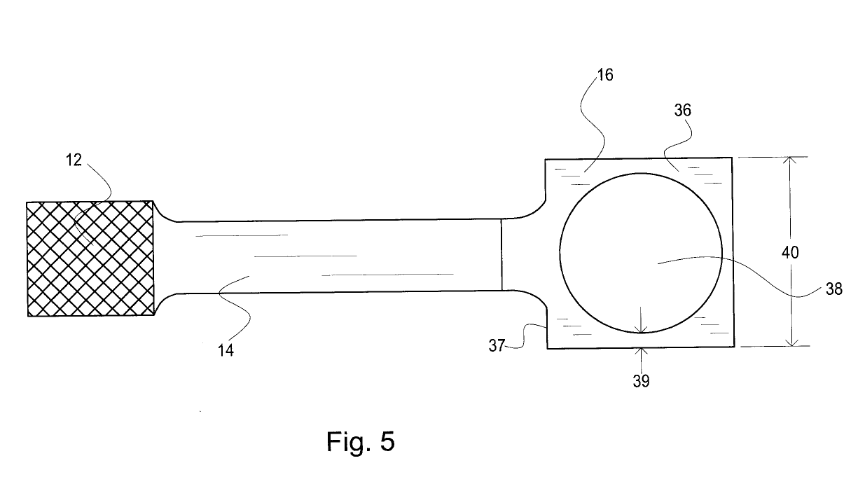

However, as best shown in FIG. 5, in one embodiment, the outer edge 37 of the attachment portion 36 may not remain substantially an equal distance from the bore 38, but the outer edge may be generally rectangularly shaped. Additionally, in one embodiment, the attachment portion may not be comprised of a bore. For example, the attachment portion may be comprised of any other urine receptacle device attachment mechanism such as, but not limited to, a clasp mechanism, as best shown in FIG. 4.

The receptacle that the holder is typically adapted to couple to is a cup. The cup typically is a specifically designed cup adapted to receive urine. The cup is typically about 1 and ¾ inches in diameter. Therefore, the bore is generally 1 and ¾ inches in diameter. There typically is a lip 43 on the upper portion of the cup which protrudes outside the outer edge of the cup past 1 and ¾ inches, allowing the cup to rest on the attachment portion of the holder when the cup is coupled to the holder, as best shown in FIG. 1B.

The receptacle 41, such as a cup, is kept substantially horizontal by using the holder in the prescribed manner. For example, since the first section and the third section are substantially parallel, if the first section is kept generally horizontal, any cup that is coupled to the third section will likewise be kept generally horizontal.

As best shown by FIG. 2, the top surface 24 of the first section 12 and the top surface 21 of the third section 16 are substantially parallel. The two surfaces are kept parallel by integrating the second section 14 at appropriate angles. For example, the top surface of the first section and the top surface 23 of the second section are generally 225 degrees apart, whereas the top surface of the third section and the top surface of the second section are generally 135 degrees apart. Generally, the plane of the second section is positioned 45 degrees relative to each of the first section plane and the third section plane.

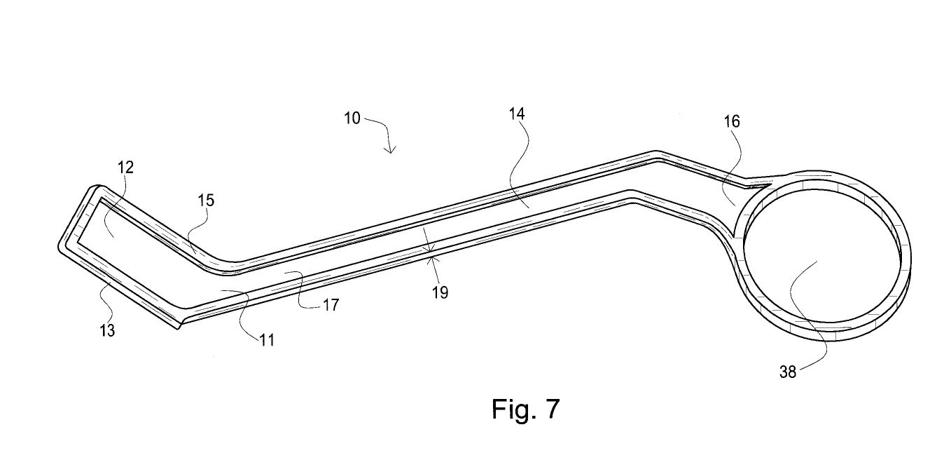

As best shown in FIG. 7, the embodiment 10 is comprised of a bottom surface 11 and one or more side surfaces 13. The one or more side surfaces are integrated to, and run between, the bottom surface and the top surfaces of the three sections. The bottom surface is typically comprised of a ridged portion 15 and an unridged portion 17. The ridged portion is typically proximal to the outer edge of the bottom surface—where the bottom surface is integrated to the side surface.

In one embodiment, the thickness 19 of the ridged portion 15 throughout each section is generally constant. Amongst sections, however, the thickness of the ridged portion may vary. For example, in one variation, the ridged portion thickness is about 0.140 inches in the first section, about 0.095 inches in the second section and about 0.110 inches in the third section. Likewise, the unridged portion 17 is also generally constant throughout each section, but may vary amongst sections. For example, the unridged portion in a first section is about 0.085 inches in the first and third sections, but may only be 0.065 in the second section. The ridge helps create a stiffer embodiment with less material thereby reducing the cost to produce a holder.

### One Method of Collecting Urine Using an Angled Urine Collection Device Holder:

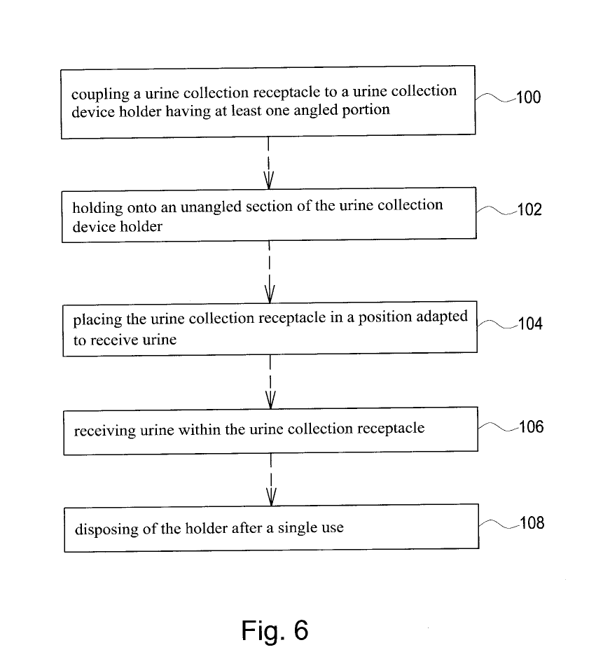

Referring to FIGS. 1 and 6, one method of collecting urine is illustrated. As shown by FIG. 1, the typical method involves using an angled urine collection device holder 10. The method may involve the collection of urine in order to perform an urination analysis on the urine upon collection. The method is adapted to allow the urine to be collected from a female subject while simultaneously allowing a collection attendant to view the urine filling the urine collection device, yet provide the female with a certain degree of privacy. Additionally, the method is adapted to allow the attendant to dispose of the urine collection device holder upon completion of the collection of the urine.

As stated, and as shown by block 100 in FIG. 6, the method comprises using an angled urine collection device holder 10. The holder typically has at least one planar second section 14 integrated angularly to at least one other planar section. One method is comprised of using a holder with two other planar sections—a first section 12 and a third section 16—which are substantially parallel and which are integrated to opposing ends of the second section.

As also shown by block 100 of FIG. 6, the method includes coupling a urine collection device to the holder. The urine collection device may be a urine collection receptacle such as a cup (i) having an outer lip and (ii) adapted to receive a lid. In other methods, the urine collection device may not be a receptacle such as a cup, but may be a different type of collection device or receptacle such as, but not limited to, a test tube. When the receptacle is a cup, the cup is typically coupled to a third section of the holder by placing the cup in a bore located in the third section.

In one method, as shown by blocks 102 and 104 of FIG. 6, a person holds onto a first section 12 of the holder and places the urine collection device in a position adapted to receive urine from the urine provider, also known as the examination subject. The method then typically includes receiving urine into the urination receptacle, as shown by block 106 of FIG. 6. Although the typical method is used in conjunction with receiving urine from a female subject, some methods may be used in receiving urine from a male subject.

In one method, the person holding onto the holder is not the person providing the urine. The person holding the holder is typically the urination analysis examination attendant, who is the person required to ensure that the urine submitted for examination is unaltered urine from the examination subject. To ensure the urine is unaltered urine, the attendant in one method will hold the holder in a position such that the receptacle may receive the urine while the attendant views the receptacle receiving the urine.

By using a holder with two unangled sections with an second section integrated to each section, the holder allows the attendant to keep the receptacle substantially parallel with the ground so as to not spill the urine. Additionally, the angled portion allows the attendant to give the urination examination subject an amount of privacy, as the attendant may be able to stand to the side of the examination subject while holding the receptacle in place with the holder, thus viewing the receptacle being filled with urine, yet giving the subject privacy as the attendant need not view the urine being released from the body.

In one method, upon collecting the urine, a lid is placed on the receptacle and the receptacle is subsequently uncoupled from the holder. Upon uncoupling the receptacle, the holder is thrown away after a single use, as shown by block 108 of FIG. 6. In another method, the receptacle may not be thrown away, but the receptacle may be placed in a recycling bin as the holder may be comprised of a polymeric material, such as, but not limited to, a recycled plastic such as Type 2 polyethylene.

### Other Embodiments and Variations:

The embodiments of the urination test receptacle holder as illustrated in the accompanying figures and described above are merely exemplary and are not meant to limit the scope of the invention. It is to be appreciated that numerous variations to the invention have been contemplated as would be obvious to one of ordinary skill in the art with the benefit of this disclosure.

I claim:

1) A unitary urination collection receptacle holder comprising:
integrated first, second and third generally planar sections, the first and third sections being generally parallel to one another with the second section spanning between the first and third sections at an angle wherein the first section is disposed above and to one side of the second section, the first section comprising a finger-hold, the third section being adapted to removeably secure a urine receptacle therein; and
wherein the urination device is comprised of a polymeric material.

2) The urination collection receptacle holder of claim 1 wherein the polymeric material comprises a commodity grade plastic.

3) The urination collection receptacle holder of claim 2 wherein, the third section includes a bore of sufficient diameter to receive the urine receptacle at least partially therein.

4) The urination collection receptacle holder of claim 2 wherein, the third section comprises a clasp, the clasp being adapted to clamp at least partially to a portion of the urine receptacle.

5) The urination collection receptacle holder of claim 2 wherein, at least a portion of one surface of the first section is textured.

6) The urination collection receptacle holder of claim 2 wherein, a plane of the second section is angled about 45 degrees relative to a plane of the first section.

7) The urination collection receptacle holder of claim 6 wherein, the plane of the second section is also angled 45 degrees relative to a plane of the third section.

8) The urination collection receptacle holder of claim 1 wherein the polymeric material substantially consists of polyethylene.

9) A combination comprising:
a urination collection receptacle holder, the holder including (i) a first section having at least a textured top surface, (ii) a second section extending from a first end thereof outwardly and downwardly from the first section, and (iii) a third section extending outwardly from a second end of the second section, the third section having at least one bore; and
a urine receptacle, the urine receptacle being removeably received in the bore.

10) The combination of claim 9, wherein,
the second section length is generally 3.5 times the length of the first section and generally 1.5 times the length of the third section.

11) The combination of claim 9 wherein the urination collection receptacle holder primarily consists of a recycled plastic material.

12) The urination collection device holder of claim 11, wherein, the recycled material comprises polyethylene.

13) The combination of claim 10 wherein, the second section is comprised of a ridged portion and an unridged portion.

14) The combination of claim 10 wherein a cross section of the second section includes (i) a first ridge located proximate a first edge of the cross section, (ii) a second ridge located proximate a second edge of the cross section, the second edge being opposite the first edge, and a web portion extending between the first and second ridges, the first and second ridges having first and second thicknesses respectively and the web portion having a third thickness, the third thickness being substantially less than the first and second thicknesses.

15. The combination of claim 14, wherein the thickness of the first and second thicknesses is between about 0.090-0.0140″, and the thickness of the web portion is between about 0.050-0.085″ with the third thickness being at least about 0.020″ thinner than either the first or second thicknesses.

16) A method of collecting urine, the method comprising:
removably coupling a urine collection receptacle to a urine collection receptacle holder, the urine receptacle holder comprising, (i) a first section having at least a textured top surface, (ii) a second section extending from a first end thereof outwardly and downwardly from the first section, and (iii) a third section extending outwardly from a second end of the second section, the third section having at least one bore and being substantially parallel to the first section;
holding onto a first section of the urine collection device holder, the first section being integrated to an end of the second section which opposes the end the first section is integrated to;
placing the urine collection receptacle in a position generally between and beneath the genitals of a person;
receiving urine within the urine collection receptacle as the person urinates.

17) The method of claim 16 wherein, the urine collection receptacle is coupled to the device holder by placing the receptacle in a bore located in a third section of the holder.

18) The method of claim 16 wherein, the urine collection device holder further comprising recycled polymeric material.

19) The method of claim 16 the method further comprising
placing a lid on the receptacle; and
disposing of the holder after a single use.

20) The method of claim 16, wherein,
the urine received within the urine collection is from a female; and
the person holding onto the first section is not the person providing the urine.
